(19) United States
(12) Patent Application Publication
(10) Pub. No.: US 2012/0157492 A1
(43) Pub. Date: Jun. 21, 2012

(54) ANTIBIOTIC DRUG

(75) Inventors: Ming-Chu Hsu, Glendora, CA (US); Chi-Hsin Richard King, Holladay, UT (US); Judy Yuan, Annandale, VA (US); Wen-Chang Chen, Su-ao Township (TW); Shan-Yen Chou, Taipei City (TW); Bo Shi, Jiangsu (CN)

(73) Assignee: TaiGen Biotechnology Co., Ltd., Taipei (TW)

(21) Appl. No.: 12/500,952

(22) Filed: Jul. 10, 2009

Related U.S. Application Data
(60) Provisional application No. 61/080,809, filed on Jul. 15, 2008.

(51) Int. Cl. A61K 31/4709; A61P 31/00; A61P 31/04; C07D 401/10

(52) U.S. Cl. 514/312; 546/155

(57) ABSTRACT

This invention relates to a malic acid salt of (3S,5R)-7-[3-amino-5-methyl-piperidinyl]-1-cyclopropyl-1,4-dihydro-8-methoxy-4-oxo-3-quinolinecarboxylic acid. Also disclosed is a method of treating bacterial infection by an effective amount of this salt.

### CROSS REFERENCE TO RELATED APPLICATIONS

This application claims priority to U.S. Provisional Application Ser. No. 61/080,809, filed Jul. 15, 2008. The content of the prior application is hereby incorporated by reference in its entirety.

### BACKGROUND OF THE INVENTION

Bacterial pathogens pose an ongoing threat to public health. Indeed, bacterial infections are increasingly difficult to treat with conventional antibiotic therapies, due to the prevalence of antibiotic-resistant bacterial strains. For example, despite the fact that turberculosis used to be readily treatable, its causative agent mycobacterium tuberculosis infects almost one third of the human population. In fact, the World Health Organization declared tuberculosis a global emergency. Antibiotic-resistant strains of bacteria are also potential agents for bioterrorism.

Thus, there is a need to develop new antibiotic drugs.

### SUMMARY

One aspect of this invention is a malic acid salt of Compound 1, i.e., 7-((3S,5R)-3-amino-5-methylpiperidin-1-yl)-1-cyclopropyl-8-methoxy-4-oxo-1,4-dihydroquinoline-3-carboxylic acid shown below:

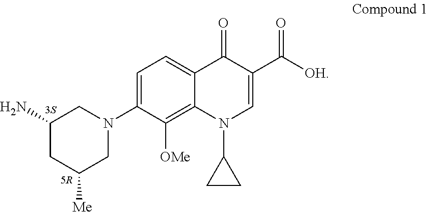

In this salt, the malic acid can be in D-malic acid, L-malic acid, or a mixture thereof and the ratio of the malic acid and Compound 1 can be 1:1.

The salt can be in solvate form, in which the salt forms a complex with a pharmaceutically acceptable solvent, e.g., water, ethanol, isopropanol, ethyl acetate, acetic acid, and ethanolamine. An example is the malic acid salt hemihydrate of (3S,5R)-7-[3-amino-5-methyl-piperidinyl]-1-cyclopropyl-1,4-dihydro-8-methoxy-4-oxo-3-quinolinecarboxylic acid.

Another aspect of this invention is a method of treating bacterial infection by the malic acid salt described above.

Also within the scope of this invention is a composition containing the malic acid salt described above and a pharmaceutically acceptable carrier for use in treating bacterial infection, as well as the use of such a composition for the manufacture of a medicament for treating bactertial infection.

Details of several embodiments of the invention are set forth in the description below. Other features, objects, and advantages of the invention will be apparent from the description, and also from the claims.

### DESCRIPTION OF THE INVENTION

One can prepare the malic acid salt described above by first synthesizing Compound 1, i.e., 7-((3S,5R)-3-amino-5-methylpiperidin-1-yl)-1-cyclopropyl-8-methoxy-4-oxo-1,4-dihydroquinoline-3-carboxylic acid, using conventional methods, and then treating this compound with malic acid. Example 1 below illustrates synthetic methods to prepare the malic acid salt.

The malic acid salt of Compound 1 thus made can be further purified by flash column chromatography, high performance liquid chromatography, crystallization, or any other suitable methods.

The above salt inhibits bacteria such as Staphylococcus aureus, Pseudomonas aeruginosa, Streptococcus pneumoniae, Enterococcus faecalis, Enterococcus faecium, Haemophilus influenzae, Escherichia coli, and Neisseria gonorrhoeae. Thus, an aspect of this invention relates to a method of treating bacterial infection by administering to a subject in need thereof an effective amount of the salt. Further, this salt can be used to treat infection caused by drug-nonsusceptible bacteria, such as methicillin-resistant Staphylococcus aureus, quinolone-resistant Staphylococcus aureus, efflux-related methicillin-resistant Staphylococcus aureus, hetero vancomycin-intermediate Staphylococcus aureus, vancomycin-intermediate Staphylococcus aureus, vancomycin-resistant Staphylococcus aureus, Penicillin-resistant Streptococcus pneumoniae, fluoroquinolone-resistant Streptococcus pneumoniae, or multi-resistant Streptococcus pneumoniae.

The term “an effective amount” refers to the amount of the active agent that is required to confer the intended therapeutic effect in the subject. Effective amounts may vary, as recognized by those skilled in the art, depending on route of administration, excipient usage, and the possibility of co-usage with other agents. The term “treating” refers to administering the active agent to a subject that has the above-mentioned infection, or has a symptom of such infection, or has a predisposition toward such infection, with the purpose to cure, heal, alleviate, relieve, alter, remedy, ameliorate, improve, or affect the infection, the symptoms of the infection, or the predisposition toward the infection. The term “nonsusceptible” used herein refers to resistance to a drug at the intermediate level through the full level. For example, methicillin-nonsusceptible bacteria can be either methicillin-resistant or methicillin-intermediate bacteria.

To practice this method, the malic acid salt can be administered orally, parenterally, by inhalation spray, or via an implanted reservoir. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

An oral composition can be any orally acceptable dosage form including, but not limited to, tablets, capsules, emulsions and aqueous suspensions, dispersions and solutions. Commonly used carriers for tablets include lactose and corn starch. Lubricating agents, such as magnesium stearate, are also typically added to tablets. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A sterile injectable composition (e.g., aqueous or oleaginous suspension) can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or di-glycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents.

An inhalation composition can be prepared according to techniques well known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art.

A topical composition can be formulated in form of oil, cream, lotion, ointment and the like. Suitable carriers for the composition include vegetable or mineral oils, white petrolatum (white soft paraffin), branched chain fats or oils, animal fats and high molecular weight alcohols (greater than C12). The preferred carriers are those in which the active ingredient is soluble. Emulsifiers, stabilizers, humectants and antioxidants may also be included as well as agents imparting color or fragrance, if desired. Additionally, transdermal penetration enhancers may be employed in these topical formulations. Examples of such enhancers can be found in U.S. Pat. Nos. 3,989,816 and 4,444,762. Creams are preferably formulated from a mixture of mineral oil, self-emulsifying beeswax and water in which mixture the active ingredient, dissolved in a small amount of oil, such as almond oil, is admixed. An example of such a cream is one that includes about 40 parts water, about 20 parts beeswax, about 40 parts mineral oil and about 1 part almond oil. Mixing a solution of the active ingredient in vegetable oil, such as almond oil, with warm soft paraffin and allowing the mixture to cool may formulate ointments. An example of such an ointment is one that includes about 30% almond and about 70% white soft paraffin by weight.

A carrier in a pharmaceutical composition must be “acceptable” in the sense that it is compatible with active ingredients of the formulation (and preferably, capable of stabilizing it) and not deleterious to the subject to be treated. For example, solubilizing agents, such as cyclodextrins (which form specific, more soluble complexes with one or more of active compounds of the extract), can be utilized as pharmaceutical excipients for delivery of the active ingredients. Examples of other carriers include colloidal silicon dioxide, magnesium stearate, cellulose, sodium lauryl sulfate, and D&C Yellow #10.

The malic acid salt of compound 1 described above can be used together with an isomeric salt, such as the malic acid salt of (3S,5S)-3-amino-5-methylpiperidin-1-yl)-1-cyclopropyl-8-methoxy-4-oxo-1,4-dihydroquinoline-3-carboxylic (Compound 1′) described in WO 2007/110836, at any ratio (e.g., 1:1). The structure of Compounds 1′ is shown below:

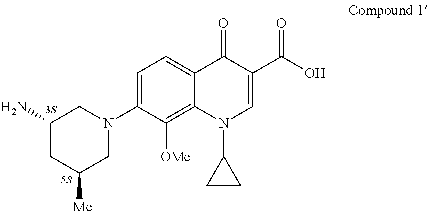

Suitable in vitro assays can be used to preliminarily evaluate the efficacy of one of the malic acid salt of Compound 1 in inhibiting growth of bacteria. The compound can further be examined for its efficacy in treating bacterial infection by in vivo assays. For example, the compound can be administered to an animal (e.g., a mouse model) having infection and its therapeutic effects are then accessed. Based on the results, an appropriate dosage range and administration route can also be determined.

Without further elaboration, it is believed that the above description has adequately enabled the present invention. The following specific examples are, therefore, to be construed as merely illustrative, and not limitative of the remainder of the disclosure in any way whatsoever. All of the publications, including patents, cited herein are hereby incorporated by reference in their entirety.

### Example 1

### Synthesis of Malic Acid Salt of Compound 1

The scheme below illustrates a synthetic route to this salt:

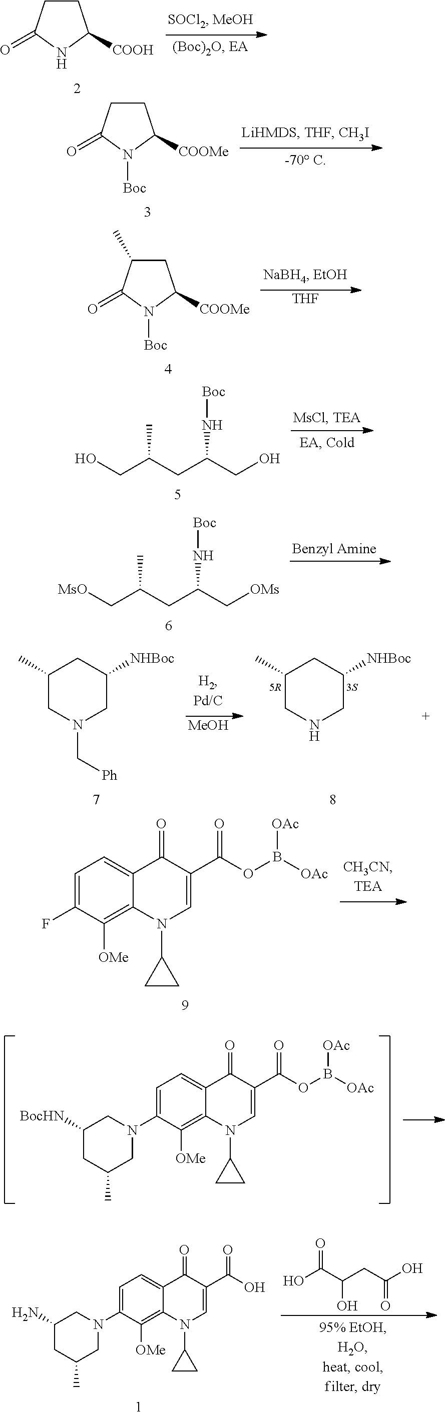

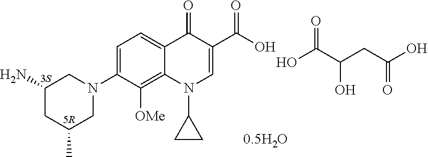

### (1) (2S)-5-Oxo-pyrrolidine-1,2-dicarboxylic acid 1-tert-butyl ester 2-methyl ester (Compound 3)

To a suspension of pyroglutamic acid (15.0 g) in methanol (60.0 mL) was added thionyl chloride (27.6 g) at <30° C. with stirring. After an hour, HPLC showed completion of the reaction. The solvent was removed under reduced pressure and the residue was dissolved in ethyl acetate (200 mL). After slow addition of triethylamine (13.5 g) at <30° C., the mixture was filtered. DMAP (1.5 g) was added to the filtrate in one portion followed by addition of Boc₂O (27.8 g) at <30° C. After HPLC showed completion of the reaction, the mixture was cooled to 0° C. and 1N HCl (13.0 mL) was added at <30° C. and stirred for 10 min. The organic layer was removed, washed with H₂O (20.0 mL), and evaporated under reduced pressure. Then, tert-butyl methyl ether (27.0 mL) was added to the obtained residue and cooled to 0° C. with stirring. The crystals that deposited slowly were filtered to give Compound 3 (21.9 g, 77.3%).

### (2) (2S,4R)-4-Methyl-5-oxo-pyrrolidine-1,2-dicarboxylic acid 1-tert-butyl ester 2-methyl ester (Compound 4)

To a 3 L flask was added a solution of Compound 3 (103.6 g) in anhydrous THF (1200.0 mL). The reaction mixture was then was cooled to −72° C. under N₂. 1.0 M LHMDS in THF (440.0 mL) was cooled to −10° C. and added to the reaction mixture at a rate to maintain the solution temperature at <−64° C. After the addition, the reaction mixture was kept <−65° C. for 45 min with stirring. Then, iodomethane (65.7 mL) was added dropwise at <−65° C., and stirred for 2 h at <−72° C. The mixture was warmed to ambient temperature and stirred for 3 h. Acetic acid (38.9 mL) in THF (300.0 mL) was added to stop the reaction. The solvent was removed under reduced pressure, H₂O (700.0 mL) and ethyl acetate (500.0 mL) was added in successively and stirred for 10 min. The aqueous layer was removed and subjected to extraction with ethyl acetate (300.0 mL). The combined organic layers were evaporated under reduced pressure to give Compound 4 (139.8 g).

### (3) (1S,3R)-4-Hydroxy-1-hydroxymethyl-3-methyl-butyl)-carbamic acid tert-butyl ester (Compound 5)

To a flask was added a solution of Compound 4 (50.0 g) in THF (400.0 mL) and the solution was cooled to 0° C. with stirring under N₂. NaBH₄ (22.0 g) was added in portions at a rate to maintain the solution temperature at the range of −5 to 5° C. and then anhydrous ethanol (100.0 mL) was added drop-wise at that temperature. The reaction mixture was kept at −5 to 5° C. for 5 h, and then warmed up to room temperature and stirring was continued overnight. When TLC showed completion of the reaction, the reaction was cooled to 5-15° C. Acetic acid (37.0 mL) was added slowly to maintain the reaction temperature at 5-15° C. until pH=5. Then H₂O (100.0 mL) was added into the mixture and stirred for 10 min and followed by ethyl acetate (150.0 mL) and stirred for 10 min. The aqueous layer was extracted with ethyl acetate (75.0 mL). The organic layers were combined into a flask. Then saturated brine (150.0 mL) was added into the flask with stirring, which then followed by sodium carbonate (14.5 g) addition to allow pH>7. The separated organic layer was washed with brine (150 mL×2) and evaporated under reduced pressure. Ethyl acetate (100.0 mL) and toluene (100.0 mL) were added to the residue and distilled under reduced pressure to give Compound 5 (41.2 g, 90.9%).

### (4) (2S,4R)-Methanesulfonic acid 2-tert-butoxycarbonylamino-5-methanesulfonyloxy-4-methyl-pentyl ester (Compound 6)

A solution of 5 (40.8 g) in ethyl acetate (347.0 mL) was cooled to 0° C. under N₂. Triethylamine (70.7 g) and methanesulfonyl chloride (59.8 mL) were added dropwise at 0±5° C. The reaction solution was stirred at 0±5° C. for 1 h. After TLC showed the completion of the reaction, a saturated sodium bicarbonate solution (350.0 mL) was added with stirring. The organic layer was removed and evaporated under reduced pressure to give Compound 6 (66.7 g, 97.9%).

### (5) (3S,5R)-3-(tert-Butoxycarbonylamino)-5-methyl-N-benzyl-piperidine (Compound 7)

Benzylamine (57.8 g) was transferred to a flask and heated to 45° C. under N₂. To the flask was added dropwise Compound 6 (65.7 g) in dimetoxyethane (65.0 mL). During the addition, the reaction temperature was maintained at about 50±5° C. After stirring overnight at the same temperature, a solution of potassium carbonate (32.8 g) in H₂O (200.0 mL) was added. The mixture was cooled to room temperature and ethyl acetate (300.0 mL) was added. The organic layer was removed, washed with H₂O (200.0 mL×2), evaporated under reduced pressure. The residue was subjected to silica gel column chromatography using 1:14 ethyl acetate/heptane as the eluant to give oily product 7.

MS (CI): 305.1;

¹H-NMR: 7.20-7.35 (m, 5), 4.27 (d, 1), 3.66 (m, 1), 3.52 (d, 1), 3.46 (d, 1), 3.05 (dd, 1), 2.73 (dd, 1), 1.97 (dd, 1), 1.74 (m, 1), 1.56 (dd, 1), 1.51 (ddd, 1), 1.41 (s, 9), 0.65 (ddd, 1), 0.84 (d, 3).

### (6) (3S,5R)-3-(tert-butoxycarbonylamino)-5-methylpiperidine (Compound 8)

A mixture of Compound 7 (4.8 g), active carbon (0.5 g), and methanol (100.0 mL) was agitated for 0.5 h and filtered. The filtrate was transferred into a hydrogenation flask and palladium on carbon (7.5%, 1.0 g) was added. The air in the flask was removed under vacuum and replaced by H₂ several times. Then the mixture was warmed to 45±5° C. and agitated under hydrogen for 36 h. The mixture was filtered, and the filtrate was evaporated under reduced pressure to give Compound 8 (2.9 g, 85.8%).

MS (CI): 215.1 (M+1);

IR: 3324, 3177, 3100-2700, 1698, 1550, 1291, 1246, 1173;

1H-NMR (300 MHz, CDCl₃): 4.30 (d, 1H), 3.40 (m, 1H), 3.20 (dd, 1H), 2.91 (dd, 1H) 2.01 (dd, 1H), 2.11 (m, 1H), 1.60 (dd, 1H), 1.51 (ddd, 1H), 1.39 (s, 9H), 0.76 (ddd, 1H), 0.82 (d, 3H);

¹³C-NMR (75 MHz, CDCl₃): 155.2, 79.3, 53.5, 51.9, 48.8, 40.8, 32.5, 28.4, 19.1.

### (7) Boron ester of 1-Cyclopropyl-7-fluoro-8-methoxy-4-oxo-1,4-dihydro-quinoline-3-carboxylic acid (Compound 9)

A reactor was charged with boron oxide (2.0 kg, 29 mol), glacial acetic acid (8.1 L, 142 mol), and acetic anhydride (16.2 L, 171 mol). The resulting mixture was refluxed at least 2 hours, and then cooled to 40° C., at which temperature, 1-cyclopropyl-7-fluoro-8-methoxy-4-oxo-1,4-dihydro-quinoline-3-carboxylic acid (14.2 kg, 51 mol) was added. The mixture was refluxed for at least 6 hours, and then cooled to about 90° C. Toluene (45 L) was added to the reaction. At 50° C., tert-butylmethyl ether (19 L) was added to introduce precipitation. The mixture was then cooled to 20° C. and filtered to isolate the precipitation. The isolated solid was then washed with tert-butylmethyl ether (26 L) prior to drying in a vacuum oven at 40° C. (50 torr) to afford Compound 9 in a yield of 86.4%.

Raman (cm⁻¹): 3084.7, 3022.3, 2930.8, 1709.2, 1620.8, 1548.5, 1468.0, 1397.7, 1368.3, 1338.5, 1201.5, 955.3, 653.9, 580.7, 552.8, 384.0, 305.8;

¹H NMR (CDCl₃, 300 MHz) δ (ppm): 9.22 (s, 1H), 8.38-8.33 (m, 1H), 7.54 (t, J=9.8 Hz, 1H), 4.38-4.35 (m, 1H), 4.13 (s, 3H), 2.04 (s, 6H), 1.42-1.38 (m, 2H), 1.34-1.29 (m, 2H).

### (8) 7-((3S,5R)-3-Amino-5-methylpiperidin-1-yl)-1-cyclopropyl-8-methoxy-4-oxo-1,4-dihydroquinoline-3-carboxylic acid (Compound 1)

A solution of compound 8 (2.0 g), Compound 9 (4.2 g), and triethylamine (3.9 mL) in acetonitrile (32.0 mL) was heated at 50° C. with stirring for at least 12 h while monitoring by HPLC. After this period, the re-cooled solution was distilled under reduced pressure. The residue was cooled to 25° C. and sodium hydroxide solution (9.3 g, 30%) was added slowly. The reaction was monitored by HPLC. After this period, the re-cooled solution was distilled under reduced pressure at 50° C. The residue was cooled to 25° C. and acetic acid (2.5 g) was added to adjust pH to 8.0. Then, dichloromethane (80.0 mL) was added with stirring. The separated organic layer was distilled under reduced pressure and hydrochloride solution (12.2 g, 30%) was added. The reaction mixture was agitated and heated to 35° C. for 12 h and monitored by HPLC. After this period, the reaction mixture was cooled to 25° C. and the precipitate was filtered. The precipitate was dissolved in water (40.0 mL) at 50° C. and neutralized with aqueous sodium hydroxide solution (3.0 g, 30%) to pH=7.9. The precipitate was filtered and dried under vacuum for 24 h at 50° C. to give Compound 1 (2.9 g, 82.9%, 99.9% purity).

### (9) 7-((3S,5R)-3-amino-5-methylpiperidin-1-yl)-1-cyclopropyl-8-methoxy-4-oxo-1,4-dihydroquinoline-3-carboxylic acid D,L-malic acid salt hemihydrate (the malic acid hemihydrate of Compound 1)

Ethanol (14 mL) and purified water (9.8 mL) were mixed and heated to 60±2° C. Compound 1 (2.8 g), dl-malic acid (1.0 g), and activated carbon (0.13 g) were added to the solution. After being stirred for 10 minutes at the same temperature, the mixture was filtered. The filtrate was cooled to 0±2° C. and stirred for 30 min to afford a precipitate, which was dried under vacuum for 2 h at 45±2° C. to give the desired salt (2.5 g, 64.7%, 98.9% purity).

MS (CI): 372.0 (M+1);

IR: −3438, 3100-2700, 1729, 1618. 1520, 1443, 1258;

¹H-NMR (300 MHz, CDCl₃): 8.67 (s, 1H), 7.63 (d, 1H), 7.15 (d, 1H), 4.24 (dd, 1H), 4.12 (m, 1H), 3.97 (m, 1H), 3.64 (s, 3H), 3.57 (dd, 1H), 3.47 (dd, 1H), 2.71 (dd, 1H), 2.69 (dd, 1H), 2.41 (dd, 1H), 2.51 (dd, 1H), 2.13 (m, 1H), 1.92 (ddd, 1H), 1.12 (ddd, 1H), 1.12, 0.90 (m, 4H), 0.90 (d, 31H);

¹³C-NMR (75 MHz, CDCl₃): 178.9, 177.6, 176.2, 169.2, 150.8, 150.3, 141.5, 136.6, 121.4, 120.0, 119.3, 105.3, 68.5, 60.3, 56.4, 52.0, 47.8, 41.6, 40.0, 36.7, 29.7, 17.8, 8.9, and 8.9.

### Synthesis of the Malic Acid Salt Hemihydrate of Compound 1′

The malic acid salt hemihydrate of Compound 1′ was prepared in a manner similar to that described in WO 2007/110836.

### Example 2

### Inhibition of Bacteria

The malic acid salts of Compound 1 and Compound 1′ and ciprofloxacin were tested for their inhibitory effect against 8 ATCC reference strains (i.e., E. coli ATCC 25922, P. aeruginosa ATCC 27853, S. aureus ATCC 29213, E. faecalis ATCC 29212, S. pneumoniae ATCC 49619, H. influenzae ATCC 49247, H. influenzae ATCC 49766, and N. gonorrhoeae ATCC 49226) and 10 clinical strains (i.e., two S. aureus strain, one E. faecalis strain, one E. faecium strain, two E. coli strains, two P. aeruginosa strains, and two H. influenzae strain).

Minimum inhibitory concentrations (MICs) were determined by the Broth microdilution method following the guidelines of the Clinical and Laboratory Standards Institute (CLSI). See, e.g., “Methods for Dilution Antimicrobial Susceptibility Tests for Bacterial That Grow Aerobically,” CLSI 2006, Approved standard-7^{th} Ed. M7-A7; and “Performance standards for Antimicrobial Susceptibility Testing,” CLSI 2007, 17^{th} Informational Supplement, M100-S17. Note that since there is no CLSI recommended broth microdilution (BMD) method for N. gonorrhoeae, the BMD method recommended for N. meningitis was used.

Each of the three compounds was dissolved in water before sterile filtration to prepare stock solutions. The stock solutions were stored at −20° C. in aliquots. Before the susceptibility testing, each stock solution was diluted in a broth medium to make a 2× stock, which had a concentration twice the final working concentration. The 2× stock was dispensed at 50 μl per well into the 96-well microtitre plates. The plates were either used fresh or stored at −80° C. before use.

To conduct the test, an aliquot of the frozen bacterial isolates was subcultured onto sheep blood agar for all species except that H. influenzae and N. gonorrhoeae were subculuted on chocolate plates. All plate media were purchased from BBL (Becton Dickinson Microbiology System, Cockeysville, Md.). The day before the antimicrobial susceptibility testing, all bacteria were sub-cultured again to obtain fresh starting cultures. When conducting the testing, Mueller-Hinton broth (MHB, Trek Diagnostics, West Essex, England) was used for E. coli, P. aeruginosa, S. aureus, and E. faecalis; MHB containing 3% lysed horse blood (MHBHB) was used for testing S. pneumoniae and N. gonorrhoeae; and Haemophilus Testing Medium broth (HTM) was used for testing H. influenzae.

On the day of testing, a 0.5 McFarland suspension of each bactrium was first prepared from a freshly subcultured plate. The 0.5 McFarland suspensions of E. coli, P. aeruginosa, S. aureus, and E. faecalis were prepared in water, while those of N. gonorrhoeae and S. pneumonia were prepared in MHB, and those of H. influenzae were prepared in HTM. One hundred microlier of each suspension was then transferred to 10 ml of MHB, MHBHB, or HTM as needed to obtain a suspension containing 1×10⁶ CFU/ml. Then, the suspension was dispensed into the 96-well microtitre plates described above (50 μl per well). All plates were incubated at 35° C. under ambient air overnight except that N. gonorrhoeae was incubated under 5% CO₂. Incubation time and condition were the same as those set forth in the guidelines of CLSI. Purity check was performed on each isolate using a loopful of the final inoculum.

The results show that the malic acid salt of Compound 1 exhibited low MICs comparable with, or even lower than, that of ciprofloxacin against the tested bacterial strains, indicating that this compound effectively inhibited bacteria. Compared to malic acid salts of Compounds 1′, the malic acid salt of Compound 1 was more efficacious in inhibiting in some S. aureus, H. influenzae, and P. aeruginosa stains.

### Inhibition of Drug-Resistant Bacteria

The malic acid salt of Compound 1, the malic acid salt of Compound 1′, and a mixture of the malic acid salts of Compound 1 and 1′ were tested for its inhibitory effect against ciprofloxacin-resistant S. aureus, and levofloxacin-resistant S. pneumoniae. MICs were determined using the broth microdilution method.

The MIC₅₀ and MIC₉₀ values of malic acid salt of Compound 1′, malic acid salt of Compound 1, a mixture of malic acid salts of Compounds 1 and 1′, ciprofloxacin (CIP), and levofloxacin (Levo) are shown in the following tables:

| | MIC₅₀ (MIC₉₀), ug/mL | | | | |
|---|---|---|---|---|---|
| | Compound | Compound | Compound | | |
| | 1′ | 1 | 1 + 1′ | | |
| Bacteria | (3S,5S) | (3S,5R) | (1:1 w/w) | CIP | Levo |
| MRSA-CIP (R) | 1.0 (2.0) | 1.0 (1.0) | 1.0 (1.0) | 64 | — |
| MRSA-CIP (S) | 0.03 (0.06) | 0.03 (0.06) | 0.03 (0.06) | 0.5 | — |
| S. pneumo- | 0.5 (2.0) | 0.25 (1.0) | 0.5 (1.0) | — | >128 |
| Levo (R) | | | | | |
| S. pneumo- | 0.06 (0.06) | 0.03 (0.06) | 0.06 (0.06) | — | 16 |
| Levo (S) | | | | | |

CIP: Ciprofloxacin
Levo: Levofloxacin
MRSA-CIP (R): Clinical isolate of MRSA-ciprofloxacin resistant strain
MRSA-CIP (S): Clinical isolate of MRSA-ciprofloxacin sensitive strain
S. pneumo-Levo (R): Clinical isolate of S. pnemoniae Levofloxacin resistant strains
S. pneumo-Levo (S): Clinical isolate of S. pnemoniae Levofloxacin sensitive strains

As shown in the above table, among the tested samples, the malic acid salt of Compound 1 and a mixture of the malic acid salts of Compound 1 and 1′ were the most effective in inhibiting ciprofloxacin-sensitive and resistant S. aureus and levofloxacin-sensitive and resistant S. pneumoniae.

### Pharmacokinetic Study

Each of the malic acid salts of Compound 1 and Compound 1′ was dissolved in 0.7% lactic acid and 3% dextrose in water at a pH about 4.5 and 2.5% L-glutamic acid in water at a pH about 5.4 to provide solutions used in this pharmacokinetic study.

Male Sprague-Dawley rats (BioLASCO Taiwan Co., Ltd., Taipei, Taiwan) weighing 300-400 g were surgically implanted with polyethylene (PE-50) cannula in the jugular vein for blood sampling while under pentobarbital anesthesia the day before the in-life phase. The rats were treated with a Compound 1 or Compound 1′ solution via intravenous injection (IV) at a dose of 2.5 mg/kg (N=3) or by oral gavage (PO) at a dose of 5 mg/kg (N=4). Dose levels were based on the free base form of the compounds. For PO study, the rats were fasted overnight with water ad libitum, and then dosed the next day. Serial blood samples were collected from animals pre-dose, at 5, 10 (IV only), 15, and 30 min, and at 1, 2, 4, 6, 8, 12, and 24 hours post-dose and heparinized plasma was recovered after centrifugation. Concentrations of the test compound in blood plasma were determined by liquid chromatography-mass spectrometry analysis (MDS SCIEX, API 3000, Applied Biosystems, CA, USA) with a lower limit of quantitation of 2 ng/mL.

Standard pharmacokinetic parameters were assessed by non-compartmental analysis using WinNonlin (Version 4.0, Pharsight, CA, USA). The area under the concentration vs. time curve from the time of dosing to infinity (AUC_{(0-inf)}) was calculated by the linear trapezoidal rule. Oral bioavailability (% F) was calculated from the dose-normalized ratio of plasma exposure following oral administration to the intravenous plasma exposure in the rats by the following equation:

% F=(AUCₚₒ/AUCᵢᵥ)×(Dᵢᵥ/Dₚₒ)×100%

where D is the dose and AUC is the area-under-the-plasma-concentration-time-curve from 0 to infinity.

For IV injection the malic acid salt of Compound 1, the terminal half-life (t_{1/2}) and the area under the curve from the time of dosing to infinity (AUC_{(0-inf)}) were 2.466±0.801 h and 1.530±0.066 μg×h/mL, respectively. For PO administration of the malic acid salt of Compound 1, the terminal half-life (t_{1/2}), the maximum of concentration (Cₘₐₓ), the time at the maximum of concentration (Tₘₐₓ), and the area under the curve from the time of dosing to infinity (AUC_{(0-inf)}) were 3.581±1.704 h, 0.622±0.170 μg/mL, 0.250±0.000 h, and 1.404±0.464 μg×h/mL, respectively.

The results show that the oral bioavailability (% F) of the malic acid salt of Compound 1 was about 45.9±15.2%, whereas the oral bioavailability (% F) of the malic acid salt of Compound 1′ was 13.2±3.3%. Thus, oral administration of the malic acid salt of compound 1 was more effective than oral administration of the malic acid salt of Compound 1′.

### Other Embodiments

All of the features disclosed in this specification may be combined in any combination. An alternative feature serving the same, equivalent, or similar purpose may replace each feature disclosed in this specification. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A malic acid salt of (3S,5R)-7-[3-amino-5-methyl-piperidinyl]-1-cyclopropyl-1,4-dihydro-8-methoxy-4-oxo-3-quinolinecarboxylic acid.

2. The salt of claim 1, wherein the ratio of the malic acid and the (3S,5R)-7-[3-amino-5-methyl-piperidinyl]-1-cyclopropyl-1,4-dihydro-8-methoxy-4-oxo-3-quinolinecarboxylic acid is 1:1.

3. The salt of claim 1, wherein the salt is in the hydrate form.

4. The salt of claim 3, wherein the salt is the malic acid salt hemihydrate of (3S,5R)-7-[3-amino-5-methyl-piperidinyl]-1-cyclopropyl-1,4-dihydro-8-methoxy-4-oxo-3-quinolinecarboxylic acid.

5. The salt of claim 4, wherein the ratio of the malic acid and the (3S,5R)-7-[3-amino-5-methyl-piperidinyl]-1-cyclopropyl-1,4-dihydro-8-methoxy-4-oxo-3-quinolinecarboxylic acid is 1:1.

6. The salt of claim 1, wherein the malic acid is D-malic acid.

7. The salt of claim 1, wherein the malic acid is L-malic acid.

8. The salt of claim 1, wherein the malic acid is D,L-malic acid.

9. The salt of claim 5, wherein the malic acid is D-malic acid.

10. The salt of claim 5, wherein the malic acid is L-malic acid.

11. The salt of claim 5, wherein the malic acid is D,L-malic acid.

12. A pharmaceutical composition, comprising the malic acid salt of (3S,5R)-7-[3-amino-5-methyl-piperidinyl]-1-cyclopropyl-1,4-dihydro-8-methoxy-4-oxo-3-quinolinecarboxylic acid and a pharmaceutically acceptable carrier.

13. The pharmaceutical composition of claim 12, further comprising the malic acid salt of (3S,5S)-7-[3-amino-5-methyl-piperidinyl]-1-cyclopropyl-1,4-dihydro-8-methoxy-4-oxo-3-quinolinecarboxylic acid.

14. The pharmaceutical composition of claim 13, wherein the ratio of the malic acid salt of (3S,5R)-7-[3-amino-5-methyl-piperidinyl]-1-cyclopropyl-1,4-dihydro-8-methoxy-4-oxo-3-quinolinecarboxylic acid and the malic acid salt of (3S,5S)-7-[3-amino-5-methyl-piperidinyl]-1-cyclopropyl-1,4-dihydro-8-methoxy-4-oxo-3-quinolinecarboxylic acid is about 1:1.

15. A method of treating microbial infection, comprising administering to a subject in need thereof an effective amount of the composition of claim 12.

16. The method of claim 15, wherein the microbial infection is infection with Staphylococcus aureus, Pseudomonas aeruginosa, Streptococcus pneumoniae, Enterococcus faecalis, Enterococcus faecium, Haemophilus influenzae, Escherichia coli, or Neisseria gonorrhoeae.

17. The method of claim 15, wherein the microbial infection is infection with methicillin-resistant Staphylococcus aureus, methicillin-resistant Staphylococcus epidermidis, quinolone-resistant Staphylococcus aureus, efflux-related Methicillin-resistant Staphylococcus aureus, hetero vancomycin-intermediate Staphylococcus aureus, vancomycin-intermediate Staphylococcus aureus, vancomycin-resistant Staphylococcus aureus, Penicillin-resistant Streptococcus pneumoniae, fluoroquinolone-resistant Streptococcus pneumoniae, or multi-resistant Streptococcus pneumoniae.

18. A method of treating microbial infection, comprising administering to a subject in need thereof an effective amount of the composition of claim 13.

19. The method of claim 18, wherein the microbial infection is infection with Staphylococcus aureus, Pseudomonas aeruginosa, Streptococcus pneumoniae, Enterococcus faecalis, Enterococcus faecium, Haemophilus influenzae, Escherichia coli, or Neisseria gonorrhoeae.

20. The method of claim 18, wherein the microbial infection is infection with methicillin-resistant Staphylococcus aureus, methicillin-resistant Staphylococcus epidermidis, quinolone-resistant Staphylococcus aureus, efflux-related Methicillin-resistant Staphylococcus aureus, hetero vancomycin-intermediate Staphylococcus aureus, vancomycin-intermediate Staphylococcus aureus, vancomycin-resistant Staphylococcus aureus, Penicillin-resistant Streptococcus pneumoniae, fluoroquinolone-resistant Streptococcus pneumoniae, or multi-resistant Streptococcus pneumoniae.
